(19) United States
(12) Patent Application Publication
(10) Pub. No.: US 2021/0165314 A1
(43) Pub. Date: Jun. 3, 2021

(54) METHOD OF CREATING A NANO-SIZED RECESS

(71) Applicant: KONINKLIJKE PHILIPS N.V., EINDHOVEN (NL)

(72) Inventors: Marcus Antonius VERSCHUUREN, Berkel-Enschot (NL); Pieter Jan VAN DER ZAAG, WAALRE (NL)

(21) Appl. No.: 16/065,307

(22) PCT Filed: Dec. 16, 2016
(86) PCT No.: PCT/EP2016/081326

§ 371 Date: Jun. 22, 2018

(30) Foreign Application Priority Data
Dec. 22, 2015 (EP) 15201885.9

(51) Int. Cl. G03F 1/38; B01L 3/00; G03F 7/00

(52) U.S. Cl. CPC G03F 1/38; B01L 3/502707; B01L 3/502761; G03F 7/0002; B01L 2200/0668; B01L 2200/0652; B01L 2200/12; G03F 7/0035

(57) ABSTRACT

The invention relates to creating a nano-sized recess into a layer of material. For that, a first layer (100) is provided, which defines a first recess (101). The first layer (100) is then conformally covered with a second layer (107) such that the second layer evenly covers the boundaries of the first recess. In this way, the second layer defines a nano-sized recess. Furthermore, the invention relates to using such a structure with a second nano-sized recess for etching a nanoslit into a graphene layer. Furthermore, such a graphene layer with a nanoslit is described to be used for creating a crossed-nanoslit device for sequencing molecules.

### FIELD OF THE INVENTION

The present invention relates to creating nano-sized recesses and nanostructures, in particular nanoslits, into a material. In particular, the present invention relates to a method of creating a nano-sized recess, to a method of creating a nanoslit into a graphene layer, to a method of creating a crossed-nanoslit device for sequencing molecules, to the use of conformal deposition for transforming a recess in a layer of material into a nano-sized recess, and to a mask layer for etching a nanoslit into a layer of material.

### BACKGROUND OF THE INVENTION

Nanopore sequencing based on a nanopore in graphene is considered to be a very promising technology for DNA and RNA sequencing as it provides a single base sequencing resolution. Furthermore, it should be able to provide long reads and it does not require complex sample preparation. Moreover, it enables a small form factor relevant for applications in point of care diagnostics, for instance in infectious disease and cancer Dx applications.

It has been discovered that cross-slit graphene devices may be used for sequencing molecules. In US 2014/0349892 A1, an apparatus and a method for the processing of single molecules, particularly for the sensing or sequencing of single-stranded DNA, are described. Hereby, a bottom layer and an electrically conductive top layer with a first and second slit, respectively, are disposed on top of each other such that an aperture is formed by the slits. The slits are preferably perpendicular to each other. Moreover, an electrical circuit may be connected to the top layer, allowing to sense single molecules that pass through the aperture.

### SUMMARY OF THE INVENTION

There may thus be a need to improve creating nano-sized recesses and nanostructures, in particular nanoslits, into a material. This is addressed by the subject-matter of the independent claims.

An aspect of the invention relates to a method of creating a nano-sized recess. The method comprises the step of providing a first layer of first material defining a first recess. Moreover, the method comprises the step of conformally depositing a second layer of second material onto the first layer such that the second material evenly covers boundaries of the first recess until the second layer defines a second nano-sized recess. Optionally, the second nano-sized recess has a minimal diameter below 5 nm.

In other words, the presented method may provide a solution for creating nano-sized recesses in that a first recess, which does not need to have a nano-sized dimension initially, may be provided in a first layer of the first material and that this first recess may be successively narrowed by conformally depositing a second layer onto the first layer and in particular onto the boundaries of the first recess. By conformally depositing the second layer onto the first layer, it may be ensured that the second material evenly covers the boundaries of the first recess. Thus, a method is presented where a first recess can be provided in a first layer with conventional methods that do not necessarily need to be capable to create nano-sized recesses and to narrow down said first recess by conformal deposition.

Such a device manufactured with the described method may for example be used as a mask layer for etching a nanostructure or more specifically a nanoslit into an underlying layer, on which the mask layer is provided. The underlying layer may for example be a conductive layer, preferably a graphene layer, a graphyne layer, a stanene layer, and/or a metallic layer. Moreover, the component manufactured with the described method may also be a supporting structure of a crossed nanoslit device, onto which a graphene layer with a nanoslit is provided. For example, the conductive layer may be an electrode, e.g. of a sequencing device.

According to an embodiment, the second nano-sized recess being a channel-like structure may be below 20 nm, below 10 nm, below 5 nm, below 3 nm, below 2.5 nm, below 2.25 nm, below 2.1 nm, below 2.0 nm, below 1.9 nm, or below 1.85 nm.

Graphynes are discussed in MALKO, D., NEISS, C., VINES, F., GÖRLING, A., Competition for Graphene: Graphynes with Direction-Dependent Dirac Cones, Physical Review Letters 108, 086804 (2012).

Stanene is discussed in ZHU, F. et. al., Epitaxial growth of two-dimensional stanene, Nature Materials 14, 1020-1025 (2015).

The material, in which the nanostructure or more specific the nanoslit is provided, may be another conductive two-dimensional material, such as a transition metal di-chalcogenides (TMDCs), or sandwich structures of graphene, two-dimensional materials and/or two-dimensional hexagonal boron nitride (which may be provided to stabilize the graphene).

A device created with the described method may for example be used for nucleic acid sequencing, e.g. of DNA, RNA, PNA, LNA, etc. Moreover such a device may be used for protein sequencing to determine the peptides composing the proteins as described in:

MOVILEANU, L., Interrogating single proteins through nanopores: challenges and opportunities, Trends Biotechnol., 2009 June; 27(6): 333-41, and

BOYNTON, P and DI VENTRA, M. Boynton, M. Di Ventra, Sequencing proteins with transverse ionic transport in nanochannels, arXiv:1509.04772 [physics.bio-ph].

The method described in this application may also be used to form planar transistors (e.g. TFTs) with a source-drain distance of a few nanometers, which source-drain distance may be formed by a single gap between the source and the drain. This gap may be created with the method described herein.

The first layer may for example be provided onto a wafer. The wafer may for example comprise a silicon (Si) layer and/or a silicon oxide (SiO₂) layer.

The first layer can for example be a nano imprint layer. Moreover, the first material of the first layer may be an electron beam resist like ZEP520 from Zeon Corp. Moreover, the first material may also be Si₃N₄. The first layer may also comprise multiple sub-layers comprising different materials.

The first recess may relate to a portion of the first layer, where some material of the first layer is removed or is not applied to. For example, the first recess may have an elongated shape. Thus, the first recess may be a first elongated recess. For example, the first recess may be structured as a first channel-like structure or trench. The boundaries of the first recess may refer to boundary surfaces of the first recess. For example, the first recess may comprise at least a side-wall and a bottom-wall. Moreover, the first recess may have a minimal diameter between 10 nm and 50 nm, preferably between 10 nm and 20 nm. Furthermore, the boundaries of the first recess may comprise at least a side-wall and a bottom-wall and the aspect ratio between the height of the side-wall and the width of the bottom-wall may be larger than 1, preferably larger than 2. According to a further exemplary embodiment, the aspect ratio may be between 1 and 3. In this way, the nano-sized recess obtained after conformal deposition may be well defined enough.

The features and characterizations that are described in respect of the first recess do also apply to other recesses described in the present application, in particular to the third recess.

The second recess may defined by the second layer deposited onto the boundaries of the first recess. Thus, the second recess may be located within the first recess. The same features and characterizations also apply to the fourth recess.

The second material may be any material that can be conformally deposited onto the first layer. In particular, the second material may be a material that can be deposited by atomic layer deposition (ALD) and/or by low pressure chemical vapor deposition (LPCVD). For example, the second material may be selected from the group consisting of SiO₂, Al₂O₃, HfO₂, TiN, TiO₂, TaN, Si₃N₄ and any combination thereof. It may have to be noted that deposited layers such as Si₃Ni₄ (silicon nitride) may not always be exactly stoichiometric under conventional deposition techniques.

Atomic Layer Deposition (ALD) may refer to a thin film deposition method in which a film is grown on a substrate by exposing its surface to alternate gaseous species (typically referred to as precursors). In contrast to chemical vapor deposition, the precursors may not be present simultaneously in the reactor, but they may be inserted as a series of sequential, non-overlapping pulses. In each of these pulses the precursor molecules may react with the surface in a self-limiting way, so that the reaction terminates once all the reactive sites on the surface are consumed. Consequently, the maximum amount of material deposited on the surface after a single exposure to all of the precursors (a so-called ALD cycle) may be determined by the nature of the precursor-surface interaction. By varying the number of cycles it may be possible to grow materials uniformly and with high precision on arbitrarily complex and large substrates. ALD is more precisely described in GEORGE, S. M., Atomic Layer Deposition: An Overview, Chem. Rev., 2010, 110 (1), pp 111-131.

Chemical vapor deposition (CVD) may refer to a chemical process which may be used to produce high quality, high-performance, solid materials. In typical CVD, the wafer or substrate may be exposed to one or more volatile precursors, which may react and/or decompose on the substrate surface to produce the desired deposit. Frequently, volatile by-products may also be produced, which are removed by gas flow through the reaction chamber. Low-pressure CVD (LPCVD) may relate to CVD at sub-atmospheric pressures. Because of the reduced pressures, unwanted gas-phase reactions may be reduced and film uniformity across the wafer or substrate may be improved.

The use of conformal deposition, for example of ALD or LPCVD, may ensure that the layer thickness of the second layer can be very tightly controlled to narrow the diameter of the initial first recess in the first layer to the desired nanoscale dimension by a simple layer deposition procedure. Subsequently, the conformally deposited second layer and in particular the second nano-sized recess may be used to create a nanoslit into a graphene layer being located under the mask layer, for example by reactive ion etching.

The second layer of second material may be deposited in such a way that it is deposited directly onto the first layer. The conformal deposition may comprise atomic layer deposition and/or low pressure chemical vapor deposition. The conformal deposition may ensure that an evenly distributed second layer of second material is deposited onto the first layer. That is, the second layer may have an even thickness, also in the region of the first recess. Since the second layer may also be evenly deposited on the boundaries of the first recess (for example the side-walls and the bottom-wall), the minimal diameter of the first recess may be diminished by twice the thickness of the second layer. This is for example shown in FIG. 1D. The same explanation also accounts to the deposition of the fourth layer described below.

The conformal deposition of the second layer may be carried out until the second layer defines a second nano-sized recess. A nano-sized recess may refer to a recess that has a minimal diameter with a dimension in the nanoscale. For example, the second nano-sized recess may have a minimal diameter below 20 nm, preferably below 10 nm, more preferably below 5 nm, more preferably below 2 nm, and most preferably below 1 nm. The minimal diameter of the second nano-sized recess may refer to a diameter of the second recess which is lying in the plane of the second layer and which measures the diameter of the second nano-sized recess along a direction, where the second recess is the narrowest. For example, if the second recess would be a channel-like structure, the minimal diameter of the second recess would be the width of the channel-like structure. Thus, the width of the second recess (nano-sized recess) being a channel-like structure may be below 20 nm, below 10 nm, below 5 nm, below 3 nm, below 2.5 nm, below 2.25 nm, below 2.1 nm, below 2.0 nm, below 1.9 nm, below 1.85 nm, below 0.5 nm, or below 0.1 nm. In this way, the size of the nano-sized recess may be small enough to be capable to measure quantum mechanical tunneling currents in transconductance in a nanostructure that is created with the mask layer described in the context of the application.

However, the nano-sized recess may have such a size that DNA is able to pass through the nanostructure (e.g. nanoslit) created with the mask layer. Double-stranded DNA, ds-DNA may have a width between 2.3 nm and 1.8 nm depending on whether it is A, B or Z-DNA. Furthermore, single-stranded DNA, ss-DNA may have a radius/width of 1.0 nm. Consequently, in order to measure a tunneling current, the central pore of the crossed-nanoslit device may have such a size that the gaps between one side of the electrode and the molecule and the other side and the molecule each have such a size that quantum tunneling occurs between the electrodes and the molecule. Therefore, the size of the nanostructure (e.g. the nanoslit) in the electrode may be slightly larger than the size which would be necessary for quantum tunneling in pure nanostructures (e.g. nanoslits).

For protein sequencing, the central pore of the crossed nanoslit slit device may be adapted in such a way that the protein can pass through the central pore. For example, the diameter of the nanopore may be between 4 nm and 20 nm. Thus each nano-sized elongated recess and/or the nanoslit in the graphene layer may have a width between 4 nm and 20 nm.

The conformal deposition may be carried out in such a way that the layer thickness of the second layer added per step amounts to 0.05 to 0.1 nm per step. In this way, a conformal deposition or conformal coating of the first layer is provided that can be carried out in a step-wise self-limiting process.

Furthermore, the boundaries of the first recess may comprise at least a side-wall and a bottom-wall and the aspect ratio between a height of the side-wall and a width of the bottom-wall may be larger than 1, preferably larger than 2. Using conformal deposition techniques, it may be ensured that also boundaries of such recesses, i.e. with aspect ratios larger than 1, may be evenly covered with the second layer such that the second recess has a minimal diameter in the nanoscale.

If the first material is a first resist, the first recess can be provided in the first layer of the first resist by imprinting or structuring the first recess into the first resist. The imprinting can for example be carried out by nano imprint lithography (NIL) and/or the structuring of the first recess into the first resist may be carried out by optical lithography and/or electron beam lithography.

The first resist may be an optical resist, e.g. if optical lithography or more specifically lithography at a wavelength of 192 nm or EUV lithography is applied for creating the first recess. If nanoimprint lithography is applied, the first resist may be a nanoimprint resist, for example an epoxy or an acrylate. In this way, a chemical reaction takes place on exposure with UV light which may result in the NIL resist being transformed from a liquid to a solid. Moreover, the first resist may be a hybrid UV-NIL resist, e.g. silicon containing organic functional matrices. Furthermore, the first resist may comprise sol-gel materials which crosslink via inorganic reactions but may also be UV assisted in the inorganic cross-link reactions.

According to an exemplary embodiment of the invention, the described method may be for creating a mask layer for etching a nanostructure, in particular a nanoslit, into a further layer, for example into a graphene layer. In this embodiment, the first recess is a first elongated recess and the second nano-sized recess is a second elongated nano-sized recess. Furthermore, the first elongated recess and the second elongated nano-sized recess extend both along a first direction.

A mask layer may refer to a layer, which is deposited onto an underlying layer which mask layer is to be partly etched away. The mask layer may comprise a material that resists to etching such that only such parts of the underlying layer are etched away where the mask layer has a recess. Such a recess in the mask layer may for example be provided by lithography techniques.

Furthermore, the etching step may be carried out in such a way that the mask layer may be removed selectively with respect to the conductive material, in which the nanostructure or nanoslit is etched. Thus, the etching method may be a gentle edging method, e.g. wet chemical etching.

Both etching and masking may refer to microfabrication techniques known to the skilled person. The second, and fifth layer described in the context of the application, which act as a mask layer, may comprise a material that is known for being suited for a mask layer (i.e. with resist to the used etching technique).

In other words, the mask layer may be an etching mask for etching the nanostructure or nanoslit into the underlying layer. For example, the etching mask may comprise such a material that it can be used for reactive ion etching (RIE) of the nanostructure or nanoslit into the underlying layer.

According to a further exemplary embodiment, the method is adapted for creating a single gap between a source and a drain in a planar transistor. The planar transistor may have the same material properties as the conductive (e.g. graphene) layer described in the context of the application. However, the source-drain material can also be a material with a higher thickness, such as multiple layer graphene, aluminum, chrome, doped silicon, titanium-silicide, in general metal silicides. Subsequently a high-K material as may be applied by ALD onto the initial gap (i.e. recess) of the transistor gate dielectric, also filling the nano-gap between the source—drain on which a gate can be applied.

A further aspect of the invention relates to a method of creating a nanoslit into a conductive layer, e.g. a graphene layer. The method comprises the step of providing a conductive layer, in particular a graphene layer. Furthermore, the method comprises the step of providing the first layer and the second layer according to a method described in the context of the application onto the conductive layer or graphene layer. Finally, the method comprises the step of etching a nanoslit into the conductive layer or graphene layer, wherein the second layer defining the second nano-sized elongated recess acts as a mask layer for etching the nanoslit into the conductive layer or graphene layer.

In other words, the device comprising the first and second layers as well as the nano-sized elongated second recess may be used as a mask layer for etching the nanoslit into the graphene layer.

The graphene layer may consist of few mono layers, preferably of 2 monolayers and most preferably of 1 monolayer.

The etching step may be carried out by reactive ion etching of the nanoslit into the graphene layer, where the second nano-sized recess acts as a mask layer for the reactive ion etching of the nanoslit into the graphene layer.

An etching technique used in the context of the application may be gas phase etching under low pressures (e.g. lower than 10 mbar) as the etch needs a high anisotropic character. An etch chamber can be chamber for Reactive Ion Etching (RIE) only or a combination of Inductively Coupled Plasma (ICP) and RIE, which allows more control over the bias (energy of the ions) and reactive species created.

Preferred materials deposited by ALD that need to be etched may be materials that easily form gas-phase species, such as SiO₂, which may be etched by fluorine chemistry, provided by CF₄, CHF₃ precursors, which can be diluted by oxygen, nitrogen, argon, hydrogen to modify the etch rate.

An effect of using a NIL layer to define the first recess may be that it is possible to tune the residual layer thickness in the second nano-sized recess from a few nanometers up to several nanometers. This thin residual layer at the bottom of the second nano-sized recess may provide a protection for the underlying graphene layer when as next step e.g. plasma activated ALD is carried out. When using optical or electron beam lithography after ALD, the graphene layer may be exposed. In ALD it can take several cycles to initiate growth and therefore the graphene may exposed to the plasma in each cycle, which may damage the graphene. With the material from the residual layer present this damage may be prevented.

A further aspect of the invention relates to a method of creating a crossed-nanoslit device for sequencing molecules. The method comprises the step of providing a third layer of a third material (for example of Si₃N₄) defining a third elongated recess extending along a second direction, the second direction being different than the first direction. Furthermore, the method comprises the step of conformally depositing a fourth layer of fourth material (for example of SiO₂) onto the third layer such that the fourth material evenly covers boundaries of the third elongated recess until the fourth layer defines a fourth elongated nano-sized recess. Finally, the method comprises the step of providing a graphene layer according to the method described before onto the third layer such that the nanoslit of the graphene layer and the fourth recess cross each other.

In the context of the present application, the crossed-nanoslit device may also be denoted as sequencing device and/or nanopore device.

The molecules may refer to nucleic acid molecules. Moreover, the molecules may also refer to proteins. In that case, peptides of the proteins may be determined with the crossed-nanoslit device.

The arrangement of the nanoslit in the graphene layer and the fourth elongated nano-sized recess in the fourth layer may be referred to as the crossed-nanoslit arrangement. The nanoslit in the graphene channel and the fourth elongated nano-sized recess may cross each other at an intersection point. At this intersection point, a nanopore may be defined. This nanopore may also be referred to as central pore.

For example, the first direction and the second direction may be perpendicular to each other. The third material may for example be Si₃N₄. In general, the third material may be a material which is under tensile stress. For example, the third material may also be a silicon- and poly-silicon-membrane. The fourth material may for example be selected from the group consisting of SiO₂, TiN, Si₃N₄, Al₂O₃, HfO₂, TaN, and any combination thereof. Regarding the fourth material, materials may be preferred that easily form gaseous species in the RIE etching step such as SiO₂, TiN and Si₃N₄.

The method of this aspect described in the present application concentrates on creating the crossed-nanoslit arrangement of the crossed-nanoslit device. Further features and aspects of crossed-nanoslit devices are described in US 2014/0349892 A1.

Furthermore, the layers described in the context of this aspect of the invention may be provided onto a wafer, which e.g. comprises a Si layer and a SiO₂ layer. The wafer, e.g. the SiO₂ layer, may further comprise a microfluidic channel. When the crossed-nanoslit arrangement is provided onto the micro fluidic channel a nanopore between the micro fluidic channel and the outside of the crossed-nanoslit device may be defined. Alternatively, the backside of the wafer may opened such that the nanopore goes from one end of the crossed-nanoslit device to another end of the crossed-nanoslit device.

With the described method an improved, i.e. easier and more reliable, way of manufacturing a sequencing device is provided. In particular, it is not necessary to create a nanopore or nanohole directly but two slits which cross each other.

By providing such a crossed-nanoslit device, it may be ensured that the device only generates a tunneling current if and when DNA (or another molecule to be sequenced) will pass through the nanopore. Moreover, measurements against zero background may be carried out with this device, i.e. no signal occurs when no DNA passes through the device. Furthermore, it may be ensured that ss-DNA cannot pass through the nanopore in a folded manner, which could preclude the detection of single bases.

According to an exemplary embodiment, the third elongated recess in the third layer may be provided by covering the third layer with a second resist, imprinting or structuring a fifth elongated recess onto the second resist, and etching the third recess into the third layer using the fifth elongated recess as a mask layer. The fifth elongated recess may be imprinted or structured onto the second resist preferably by nano imprint lithography (NIL), optical lithography, and/or electron-beam lithography.

According to an exemplary embodiment of the invention, a planar portion of the fourth layer and the second resist are removed after conformal deposition of the fourth layer and before providing the graphene layer onto the third layer. In other words, a planar portion of the fourth layer and a planar portion of the second resist may be removed.

A further aspect of the invention relates to the use of conformal deposition, preferably atomic layer deposition and/or low pressure chemical vapor deposition, for transforming a recess in a layer of material into a nano-sized recess, wherein optionally the nano-sized recess has a minimal diameter below 5 nm.

Furthermore, an aspect of the invention may relate to the use of conformal deposition for transforming a recess of a layer of material into a nano-sized recess for providing a mask layer for etching a nanoslit into a further layer.

Moreover, an aspect of the invention may relate to the use of a mask layer as described herein for creating a nanoslit into another layer of material, in particular into a conductive layer, preferably into a graphene layer, a graphyne layer or a metallic layer.

Another aspect of the invention relates to a mask layer for etching a nanoslit into a layer of material, wherein the mask layer comprises a first layer of first material defining a first elongated recess extending along a first direction. Furthermore, the mask layer comprises a second layer of second material conformally deposited onto the first layer such that the second material evenly covers boundaries of the first elongated recess and the second layer defines a second elongated nano-sized recess, wherein optionally the second elongated nano-sized recess has a minimal diameter below 5 nm. This mask layer may be obtained by a method described in the context of this application, in particular with the method of claim 8.

These and other aspects of the invention will be apparent from and elucidated with reference to the embodiments described hereinafter.

### BRIEF DESCRIPTION OF THE DRAWINGS

FIG. 1A shows a flow-chart of a method according to an exemplary embodiment of the invention.

FIGS. 1B and 1C each show a first layer according to an exemplary embodiment of the invention in a side view and in a top view, respectively.

FIGS. 1D and 1E show a first layer and a second layer according to an exemplary embodiment of the invention in a side view and a top view, respectively.

FIG. 2A shows a flow-chart for a method according to a further exemplary embodiment of the invention.

FIG. 2B shows a first layer and a second layer that are deposited onto a graphene layer according to an exemplary embodiment of the invention.

FIG. 2C shows the graphene layer with a nanoslit, wherein the first and second layers are used as a mask layer according to a further exemplary embodiment of the invention.

FIG. 3A shows a flow-chart for a method according to a further exemplary embodiment of the invention.

FIGS. 3B and 3C each show a crossed-nanoslit device according to an exemplary embodiment of the invention in a side view and a top view, respectively.

FIGS. 4A to 4I each show a layered structure resulting from an intermediate step of the method of creating crossed-nanoslit device according to an exemplary embodiment of the invention.

FIGS. 5A to 5G each show a layered structure resulting from an intermediate step of the method of creating crossed-nanoslit device according to an exemplary embodiment of the invention.

FIGS. 6A and 6B each show a layered structure resulting from an intermediate step of the method of creating crossed-nanoslit device according to an exemplary embodiment of the invention.

FIGS. 7A to 7F each show a layered structure resulting from an intermediate step of the method of creating crossed-nanoslit device according to an exemplary embodiment of the invention.

It has to be noted that the figures are not drawn to the scale. Furthermore, if the same reference signs are used in different figures, they may refer to the same or similar elements. The same or similar elements may, however, also be designated by different reference signs.

### DETAILED DESCRIPTION OF EMBODIMENTS

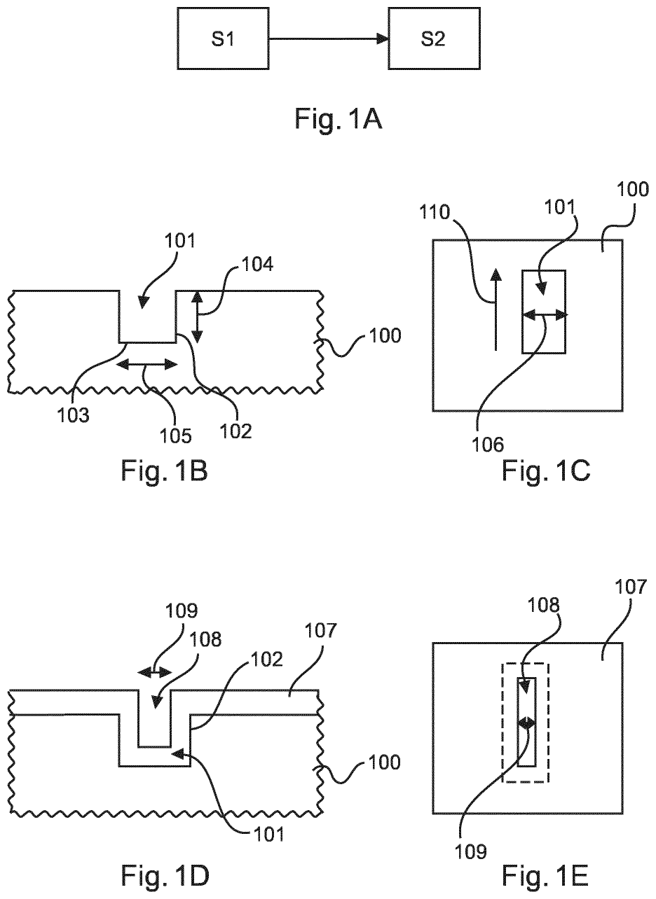

FIG. 1A shows a flow-chart of a method of creating a nano-sized recess according to an exemplary embodiment of the invention. The method comprises the first step S1 of providing a first layer of first material defining a first recess and step S2 of conformally depositing a second layer of second material onto the first layer such that the second material evenly covers boundaries of the first recess until the second layer defines a second nano-sized recess.

According to an exemplary embodiment of the invention, the second nano-sized recess has a minimal diameter below 20 nm, preferably below 10 nm, more preferably below 5 nm, more preferably below 2 nm, and most preferably below 1 nm.

According to an exemplary embodiment of the invention, the step of conformally depositing a second material comprises a method step selected from the group consisting of atomic layer deposition of the second material onto the first layer and/or low pressure chemical vapor deposition of the second material onto the first layer.

According to a further exemplary embodiment of the invention, the first recess has a minimal diameter between 10 nm and 50 nm, preferably between 10 nm and 20 nm. Furthermore, the boundaries of the first recess comprise at least a side-wall and a bottom-wall and the aspect ratio between the height of the side-wall and the width of the bottom-wall is larger than 1, preferably larger than 2.

According to a further exemplary embodiment of the invention, the step of providing a first layer comprises the steps of providing the first material which is a first resist, and of imprinting or structuring the first recess into the first resist, preferably by nano imprint lithography, optical lithography, and/or electron beam lithography.

According to a further exemplary embodiment of the invention, the first resist comprises a material selected from the group consisting of an optical resist, UV curable organic materials such as epoxies acrylates, sol-gel materials, and any combination thereof. According to a further exemplary embodiment of the invention, the second material is selected from the group consisting of SiO₂, Al₂O₃, HfO₂, TiN, TaN, Si₃N₄ and any combination thereof.

According to a further exemplary embodiment of the invention, the method is adapted for creating a mask layer for etching a nanoslit into another layer, wherein the first recess is a first elongated recess and the second nano-sized recess is a second elongated nano-sized recess. Furthermore, the first elongated recess and the second elongated nano-sized recess extend along a first direction.

FIGS. 1B and 1C each show a first layer 100 according to an exemplary embodiment of the invention which may be obtained after carrying out step Si of the method described in the context of FIG. 1A. FIG. 1B shows a side view of the first layer 100 along the first direction 110 and FIG. 1C shows a top view of the first layer 100. The first layer 100 comprises a first recess 101. In this exemplary embodiment, the first recess 101 has an elongated shape and extends along the first direction 110. In FIG. 1B it is further shown, that the first recess 101 is shaped as a channel-like structure which has side-walls 102 and a bottom-wall 103. The minimal diameter of the first recess 106 corresponds to the width of the first recess that is shaped as a first channel-like structure. In FIG. 1B it is further shown that the side-wall 102 of the first recess has a height 104 and that the bottom-wall 103 of the first recess 101 has a width 105, wherein the width 105 designates the distance between the two side-walls 102 at the bottom-wall 103. In the present case, the aspect ratio of the height 104 of the side-wall 102 and the width 105 of the bottom-wall 103 is greater than 1.

In FIGS. 1D and 1E, a first layer 100 and a second layer 107 are shown according to an exemplary embodiment of the invention, which are a result of carrying out steps S1 and S2 described in the context of FIG. 1A. It is shown that the second layer 107 conformally covers the surface of the first layer 100 including the first recess 101. As the second layer also evenly covers the side-wall 102 of the first recess 101, the second layer 107 defines a second recess 108 having a minimal diameter 109 that is diminished by twice the thickness of the second layer.

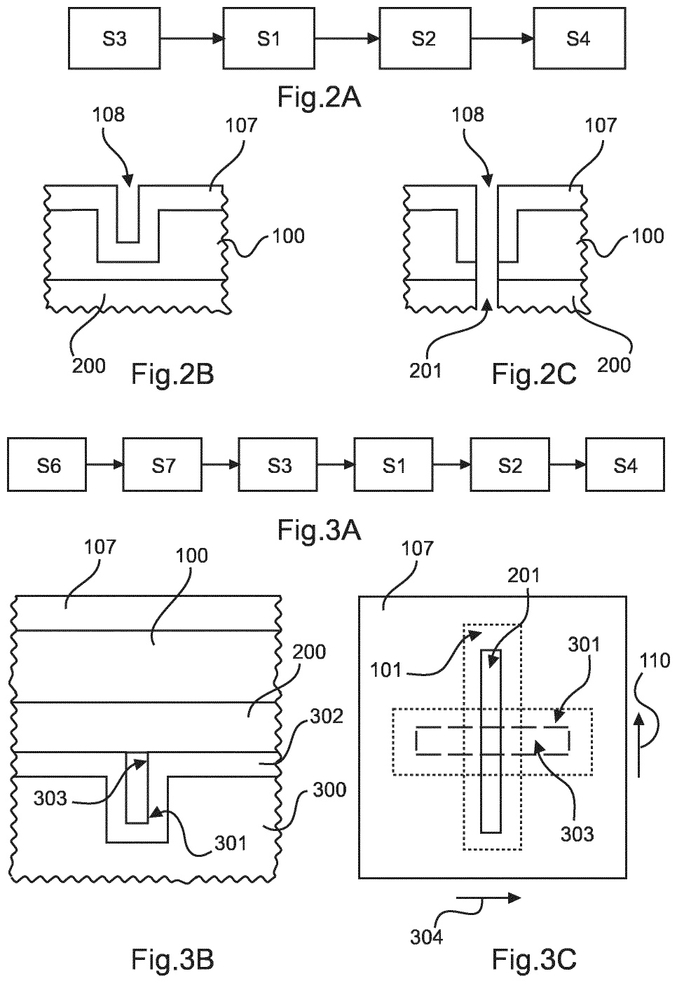

FIG. 2A shows a flow-chart of a method of creating a nanoslit in a graphene layer according to a further exemplary embodiment of the invention. The method comprises step S3 of providing a graphene layer. Subsequently, the method comprises steps S1 and S2 of providing the first and second layer as described in the context of the method shown in FIG. 1A. Moreover, the method comprises the step S4 of etching a nanoslit into the graphene layer, wherein the second layer defining the second nano-sized elongated recess acts as a mask layer for etching the nanoslit into the graphene layer.

According to an exemplary embodiment of the invention, the step of etching a nanoslit into the graphene layer comprises reactive ion etching of the nanoslit into the graphene layer, wherein the second layer defining the second nano-sized elongated recess acts as a mask layer for the reactive ion etching of the nanoslit into the graphene layer.

In FIG. 2B, a graphene layer 200 as well as a first layer 100 and a second layer 107 according to an exemplary embodiment of the invention are shown that are obtained after carrying out steps S3, S1 and S2 of the method described in the context of FIG. 2A. The graphene layer 200 is provided according to method step S3 of the method described in the context of FIG. 2A. Subsequently, first and second layers 100 and 107, wherein the second layer 107 defines a second elongated recess 108, are provided onto the graphene layer 200. In order to not overload the drawing, the first recess, which surrounds the second recess 108, is not explicitly designated.

In FIG. 2C, the graphene layer 200, the first layer 100 and the second layer 107 are shown after step S4 of the method described in the context of FIG. 2A is carried out. In method step S4, the second elongated nano-sized recess 108 is used as mask layer for etching the nanoslit 201 into the graphene layer 200. Thus, FIGS. 2B and 2C each show an etching layer 107 according to an exemplary embodiment of the invention before and after using it for etching the nanoslit 201 into the graphene layer 200.

In FIG. 3A, a flow-chart of a method for creating a crossed-nanoslit device for sequencing molecules according to a further exemplary embodiment of the invention is shown. The method comprises step S6 of providing a third layer of third material defining a third elongated recess extending along a second direction, the second direction being different than the first direction. Furthermore, the method comprises the step S7 of conformally depositing a fourth layer of fourth material onto the third layer such that the fourth material evenly covers boundaries of the third elongated recess until the fourth layer defines a fourth elongated nano-sized recess. Subsequently, the method comprises method steps S3, S1, S2 and S4 of providing a graphene layer with a nanoslit onto the third layer such that the nanoslit of the graphene layer and the fourth channel-like structure cross each other.

According to an exemplary embodiment of the invention, the third elongated recess in the third layer is provided by covering the third layer with a second resist, imprinting or structuring a fifth elongated recess onto the second resist, and etching the third recess into the third layer using the fifth layer defining the fifth elongated recess as a mask layer. According to an exemplary embodiment of the invention, the method comprises the step of removing a planar portion of the fourth layer and the second resist after conformally depositing the fourth layer and before providing the graphene layer.

In FIGS. 3B and 3C, the result that is obtained after carrying out the method described in the context of FIG. 3A is shown according to an exemplary embodiment of the invention. FIG. 3B shows a side view along the second direction 304 and FIG. 3C shows a top view. In FIG. 3B it is shown that the lowest layer is the third layer 300, which for example comprises Si₃N₄. The third layer defines a third recess 301 and is provided by method step S6 described in the context of FIG. 3A. Onto the third layer 300, a fourth layer 302 is conformally deposited such that the fourth layer evenly covers the third layer 300 and also covers the boundaries of the third recess 301. In this way, the fourth layer 302 defines a fourth nano-sized recess 303. This fourth layer 302 is obtained after a carrying out method step S7 described in the context of FIG. 3A. Onto the fourth layer 302, a graphene layer 200, a first layer 100 and a second layer 107 are provided according to method steps S3, S1, S2, and S4 as described in the context of FIGS. 3A, 2A and 1A. In FIG. 3B, the first and second recesses of the first layer 100 and the second layer 107 as well as the nanoslit in the graphene layer 200 are not shown, because they extend along the first direction 110, which is perpendicular to the second direction 304, as can be gathered from FIG. 3C.

In FIG. 3C it is further shown that the first recess 101 in the first layer and the nanoslit 201 in the graphene layer are perpendicular to the third and fourth recesses 301 and 303. Therefore, by providing such a crossed-nanoslit as shown in FIG. 3C, a nanopore emerges at the intersection point of the nanoslit 201 and the fourth recess 303.

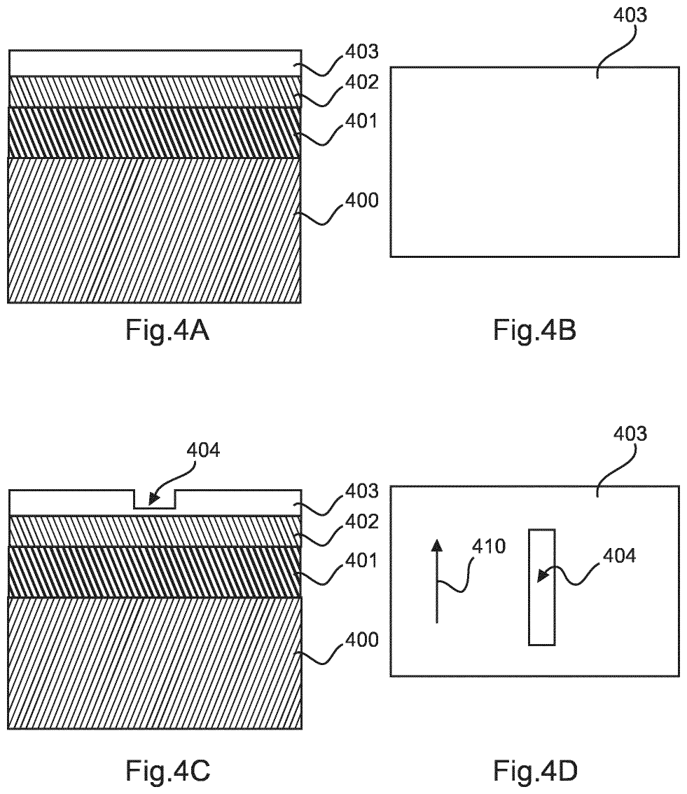

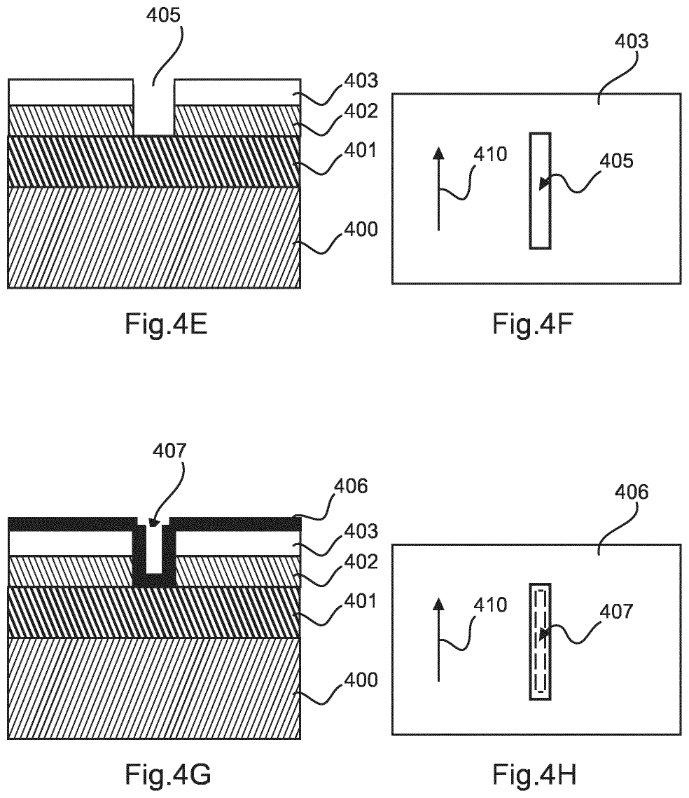

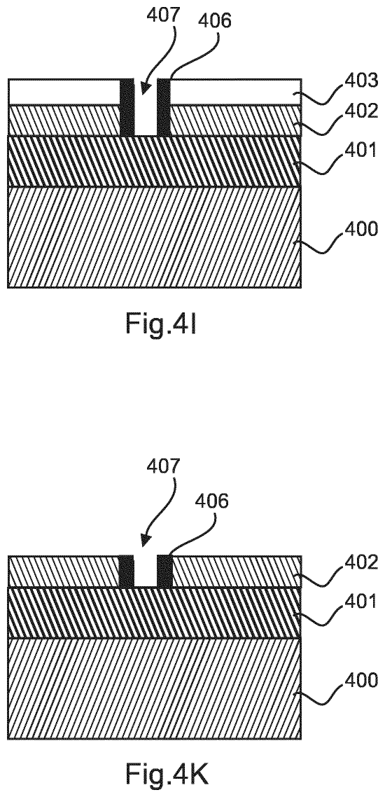

FIGS. 4A to 4I each show the result of an intermediate step of a method of creating a crossed-nanoslit device according to an exemplary embodiment of the invention. In FIG. 4A, a side view of a layered structure is shown which can be obtained after carrying out the method step S6 of the method described in FIG. 3A. FIG. 4B shows a top view of the same layered structure of FIG. 4A. The layered structure comprises a silicon layer 400 on which a silicon oxide layer 401 is located. Furthermore, using the terminology of the present application, a third layer 402, which for example is a layer of Si₃N₄, is provided on top of the silicon oxide layer 401. Furthermore, a second resist 403, for example a NIL resist, is provided on top of the third layer. As FIG. 4B shows the top view, only the second resist 403 is visible in FIG. 4B.

In FIGS. 4C and 4D it is shown in a side view and a top view that a fifth recess 404 is imprinted or structured, e.g. by nano imprinting, onto the second resist 403. According to this exemplary embodiment, the fifth recess is a fifth channel-like structure which extends along the second direction 410.

In FIGS. 4E and 4F, which show the side and top view of the same layered structure according to a further exemplary embodiment, it is shown that a third recess 405 is etched into the second resist 403 and the third layer 402. The third recess 405 is extending along the second direction 410 and is shaped as a channel-like structure.

FIGS. 4G and 4H show a side view and a top view of the same layered structure which is obtained after conformally depositing a fourth layer 406 onto the second resist 403, the third layer 402, and the third recess 405 such that the fourth layer 406 evenly covers the boundaries of the third recess. It shall be noted that the fourth layer 406 is only schematically drawn and that a conformally deposited layer may have a different shape. The layer 406 at the top of the nanoslit 407 may for example not define such edges as it is shown in FIG. 4G. In this way, the fourth layer 406 defines a fourth elongated nano-sized recess 407 which is located within the third recess 405. This fourth layer 406 may for example be conformally deposited by ALD or LPCVD and may for example comprise SiO₂.

In FIG. 4I it is shown that a planar portion of the fourth layer 406 is removed, for example by reactive ion etching such that the fourth layer 406 only covers the walls of the third channel-like structure and defines a fourth nano-sized channel-like structure 407. Furthermore, a portion of the fourth layer 406 covering the bottom of the fourth nano-sized channel-like structure 407 is removed. In FIG. 4K it is shown that the second resist 403 is removed, for example by stripping the NIL resist 403, such that only the third layer 402 remains which comprises the third recess 405 whose walls are evenly covered with the fourth layer 406 such that the fourth layer 406 defines the fourth nano-sized recess 407.

However, it shall be noted that the steps shown in FIGS. 4I and 4K may be optional. Thus, the graphene layer and the further layers may be directly applied onto the fourth layer, which may be a SiO₂ layer. Thus, it may be not necessary for removing the planar portion of the fourth layer 406 and for stripping the second resist 403.

As an alternative to the method steps shown in FIGS. 4E to 4K, the fourth nano-sized channel-like structure may also be etched into the third layer using an etching mask. In this case, the fourth layer would be conformally deposited onto the second resist 403 directly after imprinting the fifth recess 404 into the second resist 403 as it is shown in FIG. 4C. Thus, the second resist with the fourth layer conformally deposited on top, would be the etching mask. This etching mask then is used for directly etching the fourth nano-sized channel-like structure 407 into the third layer.

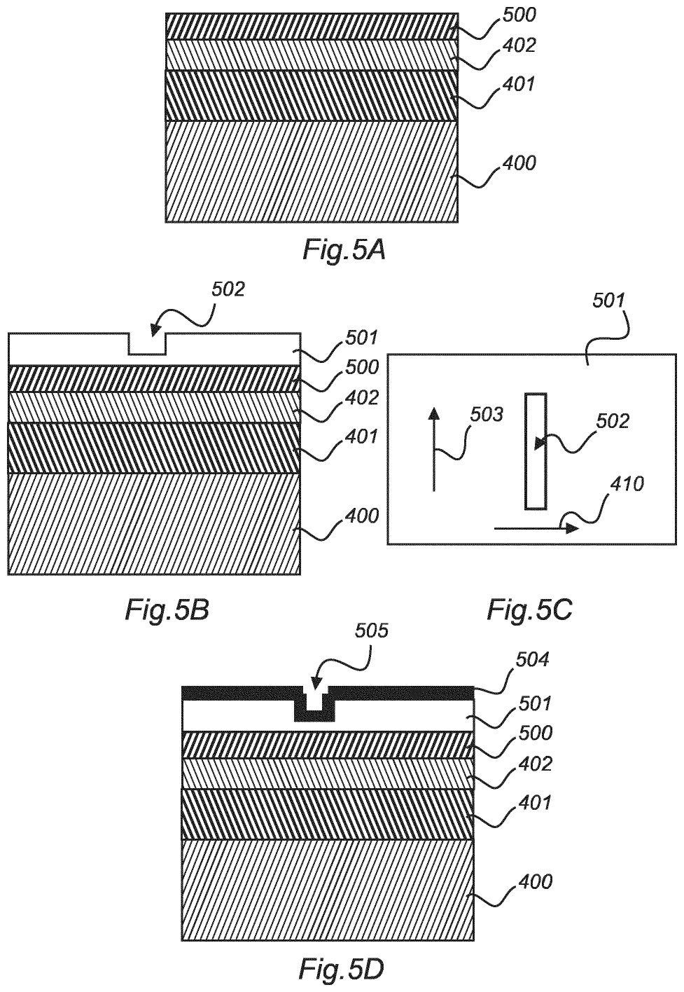

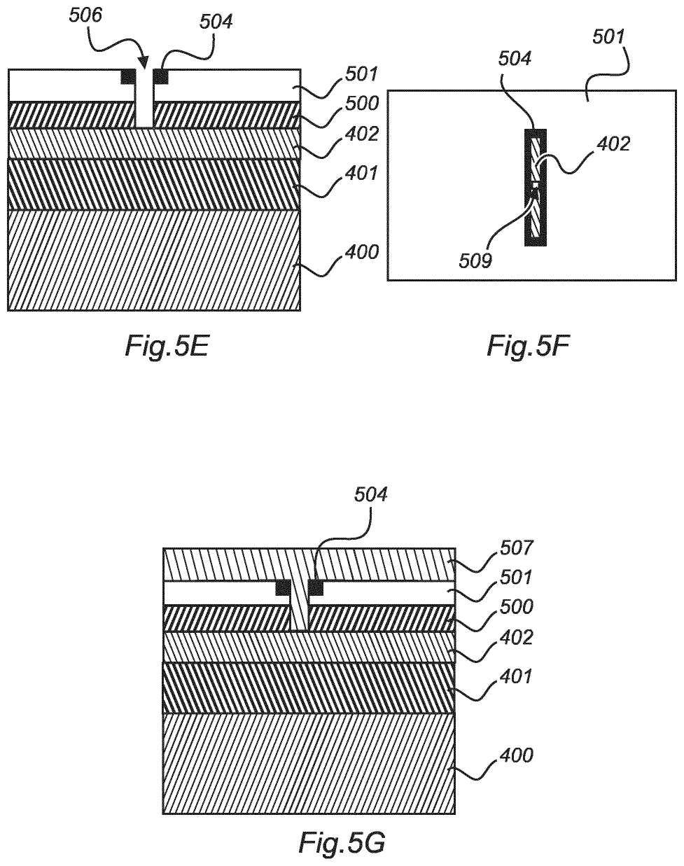

FIGS. 5A to 5G show that a graphene layer with a nanoslit is provided onto the third and fourth layers shown in FIGS. 4A to 4K according to an exemplary embodiment of the invention.

FIG. 5A shows that a graphene layer 500 is provided onto the third layer 402. It shall be noted that the graphene layer is not drawn to the scale and that it may be much thinner (e.g. a monolayer) than what is shown in FIG. 5A. In this view, the fourth recess 407 is not visible because the side view shown in FIG. 5A is along the first direction, which is perpendicular to the second direction along which the fourth nano-sized recess extends. In FIG. 5B, it is shown that a first layer 501 is provided onto the graphene layer 500, wherein the first layer 501 defines a first recess 502. The first layer 501 may be an NIL resist, wherein the first recess 502 may be imprinted by nano imprinting. FIG. 5C shows a top view of the layered structure shown in FIG. 5B. It can be seen that the first recess 502 is an elongated first recess or a first channel-like structure which extends along the first direction 503 that is perpendicular to the second direction 410.

In FIG. 5D it is shown that a second layer 504 is conformally deposited onto the first layer 501 comprising the first recess 502 such that the second layer 504 defines a second nano-sized recess 505. In this step, the high control conformal deposition, for example ALD or LPVCD, causes the first recess 502 to be narrowed by twice the thickness of the second layer 504. FIGS. 5E and 5F show a side view and a top view of the layered structure where the nanoslit 506 is etched into the graphene layer 500 and where a planar portion of the second layer 504 is removed from the first layer 501. Because the nanoslit 506 in the graphene layer 500 and the fourth nano-sized recess in the fourth layer 406 cross each other, a nanopore 509 emerges at the crossing point of the nanoslit and the fourth nano-sized recess.

In FIG. 5G, it is shown that, optionally, a further protection polymer layer 507 is provided onto the first layer 501, for example by spincoating or other process. This may however not be necessary as already the first layer 501 may protect the graphene layer 500.

Thus, the first layer 501 additionally provides a protection for the graphene layer 500. I.e. it may additionally have the effect that the buffers which may be provided in the overall sequencing device to maintain the nucleic acids (or proteins in protein sequencing) will not react or deteriorate the graphene layer (which may be a monolayer) and thereby limit the lifetime and performance of the crossed-slit nanopore device. Furthermore, it may prevent that a large shunt current through the buffer solution from one end of the crossed-slit nanopore device to the other whereby the signal measured from the tunneling (when a nucleic acid is in the nanopore device) would be overwhelmed by this background/shunting current. This is further described in paragraphs [0073] and [0074] of US 2014/0349892 A1.

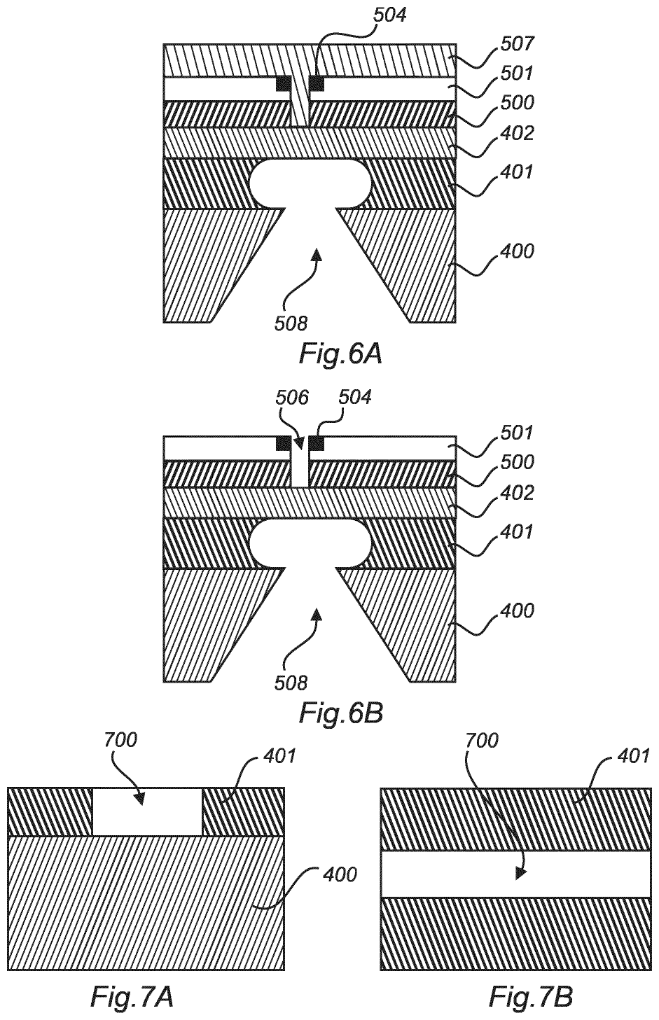

FIGS. 6A and 6B show further intermediate results when creating a crossed-nanoslit device according to an exemplary embodiment of the invention. In FIG. 6A it is shown that the back side of the silicon wafer 400 and the silicon oxide wafer 401 is opened such that a nanopore extending from one side of the first layer 501 to the other side of wafer is created. In FIG. 6B, it is shown that the polymer layer is removed, for example by washing and by supercritical drying or depolymerizing.

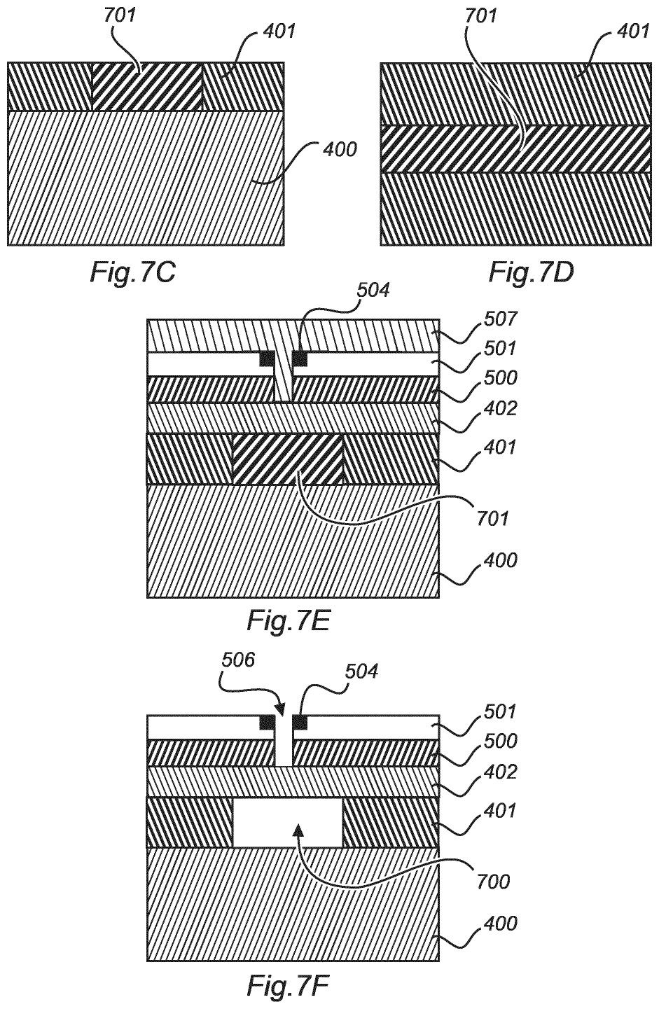

In FIGS. 7A to 7F, intermediate results of an alternative method of creating a crossed-slit device according to an exemplary embodiment are shown, where an alternative wafer is provided. In FIG. 7A it is shown that the wafer comprises a silicon layer 400 and a silicon oxide layer 401 which comprises a microfluidic channel 700. In FIGS. 7C and 7D it is shown in a side view and a top view that the microfluidic channel is filled up with a polymer 701 and that it is planarized. FIG. 7B shows a top view of the structure shown in FIG. 7A. Subsequent to providing the wafer shown in FIGS. 7A to 7D, a crossed nanoslit arrangement is provided onto the wafer as shown in FIGS. 4A to 4K and 5A to 5G.

In FIG. 7E, the layered structure is shown according to an exemplary embodiment, after the different layers are provided onto the wafer as shown in FIGS. 4A to 4K and 5A to 5G. In other words, the structure shown in FIG. 7E is the same as the one shown in FIG. 5G apart from the feature that the silicon oxide layer 401 comprises the microfluidic channel filled with the polymer 701. The polymer 701 may be a sacrificial polymer layer which is later removed, for example a layer of polymers, of PMMA, of poly-styrene, or of a cross-linked polymer that can be removed later by oxygen ions or oxygen radicals. In FIG. 7F it is shown that the polymer is removed from the top of the first and second layers 501, 504 and is also removed from the microfluidic channel 700. In this way, a nanopore emerges between the first layer and the microfluidic channel.

Further features which may be provided for creating a crossed-slit device may be described in US 2014/0349892 A1.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. Method of creating a nano-sized recess, for creating a mask layer for etching a nanoslit into an underlying layer, comprising the steps:
providing a first layer of first material defining a first elongated recess extending along a first direction; and
conformally depositing a second layer of second material onto the first layer such that the second material evenly covers boundaries of the first recess until the second layer defines a second nano-sized elongated recess extending along the first direction.

2. Method according to claim 1,
wherein the second nano-sized recess has a minimal diameter below 20 nm, below 10 nm, below 5 nm, below 3 nm, below 2.5 nm, below 2.25 nm, below 2.1 nm, below 2.0 nm, below 1.9 nm, below 1.85 nm, below 0.5 nm, or below 0.1 nm.

3. Method according to claim 1,
wherein the step of conformal depositing a second material comprises a method step selected from the group consisting of atomic layer deposition of the second material onto the first layer and/or low pressure chemical vapor deposition of the second material onto the first layer.

4. Method according to claim 1,
wherein the first recess has a minimal diameter between 10 nm and 50 nm, preferably between 10 nm and 20 nm; and/or
wherein the boundaries of the first recess comprises at least a side-wall and a bottom-wall and the aspect ratio between a height of the side-wall and a width of the bottom-wall is larger than 1, preferably larger than 2.

5. Method according to claim 4,
wherein the step of providing a first layer comprises the steps of:
providing the first material which is a first resist; and
imprinting or structuring the first recess into the first resist, preferably by nano imprint lithography, optical lithography, and/or electron-beam lithography.

6. Method according to claim 5,
wherein the first resist comprises a material selected from the group consisting of an optical resist, UV curable organic materials such as epoxies acrylates, sol-gel materials, and any combination thereof.

7. Method according to claim 6,
wherein the second material is selected from the group consisting of SiO₂, Al₂O₃, HfO₂, TiO₂, TiN, TaN, Si₃N₄ and any combination thereof.

8. Method according to claim 1,
wherein the second nano-sized recess has a minimal diameter below 5 nm.

9. Method of creating a nanoslit into a graphene layer, comprising the steps:
providing a graphene layer;
providing the first layer and the second layer according to the method of claim 8 onto the graphene layer; and
etching a nanoslit into the graphene layer, wherein the second layer defining the second nano-sized elongated recess acts as a mask layer for etching the nanoslit into the graphene layer.

10. Method according to claim 9,
wherein the step of etching a nanoslit into the graphene layer comprises reactive ion etching of the nanoslit into the graphene layer, wherein the second layer defining the second nano-sized recess acts as a mask layer for etching the nanoslit into the graphene layer.

11. Method of creating a crossed-nanoslit device for sequencing molecules, comprising the steps:
providing a third layer of a third material defining a third elongated recess extending along a second direction, the second direction being different than the first direction;
conformally depositing a fourth layer of fourth material onto the third layer such that the fourth material evenly covers boundaries of the third elongated recess until the fourth layer defines a fourth elongated nano-sized recess; and
providing a graphene layer according to the method of claim 9 onto the third layer such that the nanoslit of the graphene layer and the fourth channel-like structure cross each other.

12. Method according to claim 11,
wherein the third elongated recess in the third layer is provided by:
covering the third layer with a second resist;
imprinting or structuring a fifth elongated recess onto the second resist, preferably by nano imprint lithography, optical lithography, and/or electron-beam lithography; and
etching the third recess into the third layer using the fifth layer defining the fifth elongated recess as a mask layer.

13. Method according to claim 12, the method further comprising the step:
after conformally depositing the fourth layer and before providing the graphene layer, removing a planar portion of the fourth layer and the second resist.

14. Method according to claim 12, comprising the use of conformal deposition, preferably atomic layer deposition and/or low pressure chemical vapor deposition, for transforming a recess in a layer of material into a nano-sized recess.

15. Mask layer for etching a nanoslit into a layer of material, the mask layer comprising:
a first layer of first material defining a first elongated recess extending along a first direction; and
a second layer of second material conformally deposited onto the first layer such that the second material evenly covers boundaries of the first recess and the second layer defines a second elongated nano-sized recess.
